(19) United States
(12) Patent Application Publication
(10) Pub. No.: US 2021/0077011 A1
(43) Pub. Date: Mar. 18, 2021

(54) Electrode System for Wearable Devices

(71) Applicant: Google LLC, Mountain View, CA (US)

(72) Inventors: Julian Maclaren, Menlo Park, CA (US); Kurtis Robert Gross, Berkeley, CA (US); Christopher Workman, Mountain View, CA (US); Rafeed A. Chaudhury, San Francisco, CA (US); Vahe Tshitoyan, Fremont, CA (US); Kelly Elizabeth Dobson, Mountain View, CA (US)

(21) Appl. No.: 17/004,394

(22) Filed: Aug. 27, 2020

(30) Foreign Application Priority Data
Sep. 13, 2019 (US) PCT/US2019/051087

(51) Int. Cl. A61B 5/00; A61B 5/053

(52) U.S. Cl. CPC A61B 5/441; A61B 5/6831; A61B 2562/182; A61B 5/053; A61B 5/6804

(57) ABSTRACT

A wearable device includes at least one attachment member and an electrodermal activity (EDA) sensor comprising an integrated electrode pair physically coupled to the at least one attachment member. The electrodermal activity sensor is configured to provide an EDA signal in response to contact between the integrated electrode pair and a skin surface of a user. The integrated electrode pair includes at least two concentric electrodes radially separated by at least one insulator. Each of the at least two concentric electrodes includes an upper surface configured to contact the skin surface of the user in order to generate the EDA signal.

### FIELD

The present disclosure relates generally to wearable devices including sensors for measuring electrodermal activity (EDA) associated with users of the wearable devices.

### BACKGROUND

Wearable devices integrate electronics into a garment, accessory, container or other article worn or carried by a user. Many wearable devices include various types of sensors integrated within the wearable device to measure attributes associated with a user of the wearable device. By way of example, wearable devices may include heart-rate sensors that measure a heart-rate of a user and motion sensors that measure distances, velocities, steps or other movements associated with a user using accelerometers, gyroscopes, etc. An electrocardiography sensor, for instance, can measure electrical signals (e.g., a voltage potential) associated with the cardiac system of a user to determine a heart rate. A photoplethysmography and other optical-based sensor can measure blood volume to determine heart rate. More recently, the incorporation of sensors such as electrodermal activity (EDA) sensors into wearable devices has been proposed for measuring electrical characteristics associated with a user's skin. Characteristics such as a sympathetic nervous response, galvanic skin response (GSR), sympathetic skin response, skin conductance, etc. may be referred to as electrodermal activity and be measured using EDA sensors.

Many EDA sensors utilize electrolyte gels that can facilitate ionic flow at the skin surface of a user in order to ensure a reliable electrical contact between EDA electrodes and the user's skin. Other EDA sensors may utilize dry electrodes to measure electrodermal activity in an effort to better integrate the electrodes into the wearable device. It may be difficult, however, to maintain consistent mechanical and electrical contact between a dry EDA electrode and the surface of a user's skin. Inconsistent contact can result in noise in an EDA signal generated by the EDA sensor. Such difficulties can be exacerbated in wearable devices that are intended to provide continuous monitoring of physiological characteristics of a user wearing the device.

Accordingly, there remains a need for improved wearable devices including sensors for measuring characteristics of a user.

### SUMMARY

Aspects and advantages of embodiments of the present disclosure will be set forth in part in the following description, or may be learned from the description, or may be learned through practice of the embodiments.

One example aspect of the present disclosure is directed to a wearable device including at least one attachment member and an electrodermal activity (EDA) sensor including an integrated electrode pair physically coupled to the at least one attachment member. The electrodermal activity sensor is configured to provide an EDA signal in response to contact between the integrated electrode pair and a skin surface of a user. The integrated electrode pair includes at least two concentric electrodes radially separated by at least one insulator. Each of the at least two concentric electrodes includes an upper surface configured to contact the skin surface of the user in order to generate the EDA signal and a lower surface coupled to the at least one attachment member.

Another example aspect of the present disclosure is directed to an electrodermal activity (EDA) sensor that includes an inner electrode and an outer electrode concentric with the inner electrode. The inner electrode includes a lower surface and an upper surface separated by at least one sidewall. The outer electrode has a larger radius than the inner electrode and includes a lower surface and an upper surface separated by at least one sidewall. The EDA sensor includes one or more insulating materials concentric with the inner electrode and the outer electrode. The one or more insulating materials radially separate the inner electrode from the outer electrode and electrical insulate the inner electrode from the outer electrode. The EDA sensor includes sensing circuitry communicatively coupled to the inner electrode and the outer electrode. The sensing circuitry is configured to generate an EDA signal in response to contact between an outer surface of the inner electrode and a skin surface of a user and contact between an outer surface of the outer electrode and the skin surface of the user.

Yet another example aspect of the present disclosure is directed to a method of determining electrodermal activity (EDA) associated with a user of a wearable device. The method includes generating an EDA signal in response to contact between an integrated electrode pair and a skin surface of a user. The integrated electrode pair includes at least two concentric electrodes radially separated by a least one insulator. Each of the at least two concentric electrodes includes an upper surface that is configured to contact the skin surface of the user in order to generate the EDA signal and a lower surface that is coupled to at least one attachment member. The method includes determining a measure of electrodermal activity associated with the user in response to the EDA signal.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussion of embodiments directed to one of ordinary skill in the art are set forth in the specification, which makes reference to the appended figures, in which:

FIG. 1 is a perspective view depicting a wearable device comprising an electrodermal activity sensor having an integrated electrode pair in accordance with example embodiments of the present disclosure:

FIG. 2 is a perspective view of an electrodermal activity sensor comprising an integrated electrode pair having an inner concentric electrode and an outer concentric electrode in accordance with example embodiments of the present disclosure;

FIG. 3 is a block diagram depicting a polar circle pair of electrodes;

FIG. 4 is a block diagram depicting a parallel bar pair of electrodes;

FIG. 5 is a cross-sectional view depicting an integrated electrode pair in accordance with example embodiments of the present disclosure;

FIG. 6 is a perspective view depicting a wearable device comprising an electrodermal activity sensor having an integrated electrode pair in accordance with example embodiments of the present disclosure;

FIG. 7 is a perspective view of an electrodermal activity sensor comprising an integrated electrode pair having an inner concentric electrode and an outer concentric electrode in accordance with example embodiments of the present disclosure

FIG. 8 depicts a block diagram of a wearable device within an example computing environment in accordance with example embodiments of the present disclosure;

FIG. 9 is a perspective view depicting a wearable device comprising an electrodermal activity sensor having an integrated electrode pair in accordance with example embodiments of the present disclosure;

FIG. 10 depicts various examples of wearable devices comprising an electrodermal activity sensor in accordance with example embodiments of the present disclosure; and

FIG. 11 depicts various components of an example computing system that can be implemented as any type of client, server, and/or computing device as described herein.

### DETAILED DESCRIPTION

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations.

Generally, the present disclosure is directed to wearable devices that include sensor systems configured to measure electrodermal activity (EDA) associated with users of the wearable devices. More particularly, systems and methods in accordance with example embodiments are provided for measuring the electrodermal activity associated with a user using a sensor including concentric EDA electrodes attached to or otherwise integrated with a wearable device. By way of example, an integrated electrode pair can include at least two concentric electrodes separated by at least one insulating layer. Each of the concentric electrodes can include a three-dimensional structure having an upper surface that is configured to contact the skin of a user of the wearable device. When the upper surface of the integrated electrode pair is in contact with the skin of a user of the wearable device, the sensor can generate an EDA signal that is indicative of the electrodermal activity associated with the user. The integrated electrode pair can include one or more sidewalls (e.g., substantially vertical sidewalls) that connect to a lower surface of the integrated electrode pair. The lower surface of the integrated electrode pair can be physically coupled to an attachment member of a wearable device. In example embodiments, the EDA signal may be indicative of a measurement of conductance or resistance associated with one or more dermal layers of the user's skin. The user's perspiration can provide electrical contact between the integrated electrode pair and the skin surface of the user, thereby enabling ionic flow between the electrodes and the skin surface. A conductance or resistance associated with one or more dermal (e.g., subdermal) layers of the user's skin can then be determined using the integrated electrode pair. The EDA signal may be analyzed to determine a measured conductance or resistance in some examples. For instance, a current through the user's skin as coupled through the integrated electrode pair can be measured and correlated to determine conductance, resistance, or another measure indicative of sympathetic nervous system activity.

By utilizing a set of concentric electrodes, a more uniform sampling distribution area adjacent to the upper surface of the at least one insulating layer can be provided. The surface area of the user's skin at the electrode gap where the insulating layer separates the concentric electrodes may not be directly dependent on electrode pressure in some embodiments. A larger and more even distribution of sudoriferous glands can be sampled through a concentric design as described in order to increase the signal-to-noise ratio associated with the EDA signal. Additionally, due to the buildup of bodily fluids such as perspiration under the electrodes, small movements may not result in movement of the electrode pair to an area of dry skin. For typical pressures, the area of skin may stay constant. In this manner, increased electrode pressure may not affect the diameter of the contact patch between the electrode and the skin surface, which otherwise may affect the signal to noise ratio of the EDA signal. Furthermore, the use of concentric electrodes in some examples provides a rotation invariant design such that small band rotations may not result in changes to the EDA signal. Additionally, a single integrated electrode pair can provide a smaller form factor that can be more aesthetically pleasing and suitable for a wearable device.

Traditionally, devices that are configured to measure electrodermal activity utilize electrolyte gels that can facilitate ionic flow at the skin surface of a user in order to ensure a reliable electrical contact between EDA electrodes and the user's skin. For consumer wearable devices such as garments (e.g., jackets, pants, etc.), accessories (wristbands, bracelets, rings), containers (e.g., backpacks, purses, etc.), the use of such electrolyte gels may be impractical. Accordingly, wearable devices may utilize dry electrodes to measure electrodermal activity. A challenge with dry electrodes, however, can be maintaining consistent mechanical and electrical contact with the surface of a user's skin. If contact is not consistent, and instead varies with time, the variation may appear as spurious changes in the electrodermal activity signal, which in some instances may not be easily separated from true changes in electrodermal activity. Moreover, dry electrodes may require a minimum period of time to reach equilibrium after being placed on a user's skin, and thereby allow reliable measurement of electrodermal activity. In some instances, this settling time can be on the order of minutes. Motions can dislodge the electrodes such that the settling process may need to be restarted to provide an accurate measurement. Thus, such traditional implementations can prevent or make it difficult to continuously monitor electrodermal activity in many instances. These issues can be particularly problematic in wrist-mounted wearable devices where the device can undergo significant motion when worn by a user.

The goal of electrodermal activity monitoring can be to measure the buildup and excretion of perspiration from eccrine sudoriferous glands (also referred to as sweat glands) as an indicator of sympathetic nervous system (SNS) activity to make inferences about human psychophysiological states. During SNS activation, eccrine sudoriferous glands may excrete perspiration causing skin conductance changes. The signal caused by SNS activation can therefore be measured more effectively by summing the signal across a larger number of sudoriferous glands. Consider an example of a traditional approach that includes two circular electrodes that are separated in a lateral direction. An interface between each electrode and a user's skin may sample sudoriferous glands near the electrode pair centerline, which can result in less effective sampling further from the centerline. Other examples may utilize a set of longer parallel electrodes which may be able to more evenly sample sudoriferous glands between the two electrodes, and thereby increase the coverage of sampled sudoriferous glands. However, such implementations may result in inconsistent contact with the skin. For example, the electrode to skin contact may change with time, due to small rotations of the electrode, and/or due to small pressure changes in these type of systems.

In accordance with example embodiments of the disclosed technology, an integrated electrode pair comprising a set of two or more concentric EDA electrodes is provided. According to some aspects, the integrated electrode pair can measure the electrodermal activity of a user more evenly, effectively, and/or practically using wearable devices that includes a set of concentric EDA electrodes. The integrated electrode pair can provide a more even sudoriferous gland sampling distribution area, while also increasing the coverage area of sampled sudoriferous glands. Additionally, a set of concentric EDA electrodes can provide such benefits while measuring electrodermal activity over a more consistent area of a user's skin.

According to some implementations, an integrated electrode pair can include an inner electrode and an outer electrode that are separated by an insulating layer. The insulating layer can provide radial separation between the inner electrode and the outer electrode. The insulating layer can extend circumferentially around at least a portion of the inner electrode, and the outer electrode can extend circumferentially around at least a portion of the insulating layer. In this manner, an electrode gap is formed in a circumferential direction that matches the outer edge of the inner electrode and the inner edge of the outer electrode. In some examples, the inner electrode is laterally surrounded in its entirety by the outer electrode. The inner and outer electrodes can be laterally separated by the insulator. Accordingly, the conductance or resistance of the user's skin can be measured based on current flows extending radially from the inner electrode to the outer electrode (and/or vice versa). This can be contrasted with approaches that use two electrodes (bars or circles) that are separated linearly, in which case the conductance or resistance is measured using a current that flows in a linear direction relative to the spacing between electrodes.

The inner electrode and outer electrode can be formed with various shapes and dimensions in example implementations. Generally, the inner electrode and the outer electrode are concentric objects. Such objects may also be referred to as coaxial objects as they share the same center or axis. Circles, cylinders, polygons, spheres, polyhedra, and other shapes can be concentric with one another. In some examples, the inner electrode, the outer electrode, and the insulating layer may have a generally circular cross-sectional shape. For instance, the inner electrode, the outer electrode, and the insulating layer may have a generally cylindrical shape, with the radius of the insulating layer being larger than the radius of the inner electrode, and the radius of the outer electrode being larger than the radius of the insulating layer. As a specific example, the inner electrode can have a generally cylindrical shape and the insulating layer can have a generally cylindrical shape with a cylindrical opening in the center or middle of the cylinder to accommodate the inner electrode. In this manner, the insulating layer can extend circumferentially around the inner electrode. Similarly, the outer electrode can have a generally cylindrical shape with a cylindrical opening in the center or middle of the cylinder to accommodate the inner electrode and insulating layer. In this matter, the outer electrode can extend circumferentially around the insulating layer which can then radially separate the outer electrode form the inner electrode.

Other shapes may be utilized for the inner electrode, the insulating layer, and/or the outer electrode. Generally, any set of two or more concentric objects can be used to form the two electrodes of the integrated electrode pair. By way of example, the electrodes can be formed using spherical-shaped objects, prism-shaped objects (e.g, triangular prism, rectangular prism, or other polygonal prism), cylindrical-shaped objects, etc. It is noted that the electrodes need not have a regular polygonal or polyhedral shape. By way of example, the inner electrode can be formed in a generally cylindrical shape with an outer edge that forms a cloverleaf or other pattern. The outer edge can include multiple arc sections that curve in different directions to increase the length of the outer edge of the inner electrode. By increasing the length of the outer edge of the inner electrode, the sampling area of the user's skin can be increased. The insulating layer can be formed in a generally cylindrical shape with an opening in the middle. The inner edge and the outer edge of the insulating layer can form a matching cloverleaf pattern having a larger radial dimension(s) relative to the inner electrode. The outer electrode can be formed with an inner edge that forms a matching cloverleaf pattern with a larger radial dimension(s) relative to both the inner electrode and insulating layer. The inner edge of the outer electrode can be a continuous inner edge. The outer edge of the inner electrode can be a continuous outer edge. The outer edge of the outer electrode may have a circular shape or other shape (e.g., square, rectangle). The utilization of a cloverleaf or other pattern may increase the sampling area of the user's skin across which a conductance and/or resistance can be measured. Additionally, the use of concentric electrodes with continuous edges enables the generation of an EDA signal based on current flow between the continuous outer edge of the inner electrode and the continuous inner edge of the outer electrode. This current flow may extend radially with an even distribution between electrodes in some embodiments.

Various materials may be utilized to form the electrodes and the insulating layer(s) of the integrated electrode pair in various embodiments. In some examples, the electrodes may include one or more metallic materials. The electrodes can be formed from one or more metals and/or metal alloys. As a specific example, the inner electrode and outer electrode may be formed from silver or a silver alloy such as silver chloride. In some examples, the electrodes may include one or more conductive polymers, semiconductors, or other conductive materials. It will be appreciated that any suitable conductive material may be utilized. The insulating layer can be formed from one or more electrically insulating materials that can electrically insulate the inner electrode from the outer electrode. Any suitable electrically insulating material having high resistivity such as air or space (e.g., a vacuum), nonconductive materials (e.g., plastics, nylons, ceramics, etc.), or semiconducting materials in relatively nonconductive states can be used.

The integrated electrode pair can include a lower surface and an upper surface that define a vertical dimension of the integrated electrode pair. In some embodiments, the upper surface of the outer electrode and the upper surface of the inner electrode can be coplanar. The insulating layer can also be coplanar with the inner electrode and the outer electrode in some embodiments. In such examples, the upper surface of the inner electrode, the outer electrode, and the insulating layer can form a common upper surface of the integrated electrode pair that can contact the user's skin. In some examples, the upper surface of the integrated electrode pair can be substantially flat. In other examples, the upper surface of the integrated electrode pair can include a curve. In some instances, the upper surface of the inner electrode and/or outer electrode may include one or more raised portions to enhance contact between the electrode and the skin surface of the user. In some embodiments, the upper surface of the outer electrode and the upper surface of the inner electrode can be coplanar, while the upper surface of the insulating layer includes a vertical offset from the upper surface of the inner electrode and the outer electrode. In some examples, the upper surface of the insulating layer can be recessed relative to the upper surface of the electrodes. In some instances, such an approach may provide an enhanced electrical connection between the electrodes and the user's skin via perspiration that can build up within the recessed area. In other examples, one or more of the inner electrode and/or the outer electrode can be recessed relative to the upper surface of the insulating layer. Such an approach may be useful in situations where the buildup of perspiration may result in an inadvertent short between the inner electrode and outer electrode.

According to some example implementations, an integrated electrode pair can be physically coupled to or otherwise integrated with a wearable device to provide a non-obtrusive and effective sensor system for measuring electrodermal activity associated with a user. A wearable device may include an attachment member that physically couples the integrated electrode pair to the wearable device. By way of example, the integrated electrode pair can include a lower surface that is physically coupled to an attachment member such as the band of the wearable device, and an upper surface that is configured to contact the surface of the user's skin in order to measure conductance and/or resistance associated with the skin. The lower surface of the integrated electrode pair can be directly coupled to the band of the wearable device in example embodiments. In other examples, the lower surface of the integrated electrode pair can be physically coupled to the band or other portion of the wearable device via one or more intervening members. For example, a pressure exertion member can be mounted between the band of a wearable device and the lower surface of the integrated electrode pair in example embodiments. In this manner, the pressure exertion member (e.g., spring, compressible material, etc.) can exert a pressure on the lower surface of the integrated electrode pair and thereby cause a corresponding pressure at the upper surface where the integrated electrode pair contacts the surface of the user's skin. In some examples, multiple pressure exertion members may be used. For instance, a first pressure exertion member may be coupled between the lower surface of the inner electrode and the attachment member and a second pressure exertion member may be coupled between the lower surface of the outer electrode and the attachment member. Other intervening members may be utilized to couple the integrated electrode pair to a wearable device in example embodiments.

Any type of wearable device including hard goods and soft goods may be formed with an integrated electrode pair in accordance with example embodiments. By way of example, a wearable device may include a wristband, watch, belt, or other device intended to encircle a user's wrist or other body part. In other examples, a wearable device may be attached to a substrate of a wearable device in other manners, for example, to the arm of a sleeve or a leg of a pant. A wearable device may include a wearable garment, garment accessory, or garment container. An integrated electrode pair can be physically coupled to the band such as a waistband, wristband, headband, or other attachment member of a user wearable garment (e.g., shorts, undergarment) or to another portion of a wearable device such as a shoe. In some examples, a wearable device may include hard goods such as jewelry (e.g., ring, bracelet) or other articles having a generally stiff or non-flexible substrate.

In accordance with some example aspects, a sensor system for a wearable device may include an integrated electrode pair communicatively coupled to sensing circuitry that is configured to generate an electrodermal activity (EDA) signal that is representative of sympathetic nervous system activity of a user. The EDA signal can include or otherwise be indicative of a measurement of conductance or resistance associated with the user's skin as determined using a circuit formed with the integrated electrode pair. By way of example, the sensing circuitry and integrated electrode pair can cause (e.g., via induction or other means) a current to flow through one or more dermal layers of a user's skin. The current can be passed from one electrode into the user's skin via an electrical connection facilitated by the user's perspiration or other fluid. The current can then pass through one or more dermal layers of the user's skin and out of the skin and into the other electrode via perspiration between the other electrode and the user's skin. The sensing circuitry can measure a buildup and excretion of perspiration from eccrine sudoriferous glands as an indicator of sympathetic nervous system activity in some instances. For example, the sensing circuitry may utilize current sensing to determine an amount of current flow between the concentric electrodes through the user's skin. The amount of current may be indicative of electrodermal activity. The wearable device can provide an output based on the measured current in some examples. In some examples, a visual, audible, or haptic output can be provided by the wearable device. In other examples, data indicative of EDA activity may be transmitted to a remote computing device.

In some examples, a wearable device may include additional components such as a power source, processing circuitry, memory, input/output devices, and/or network interfaces. It is noted, however, that a wearable device may include relatively simple sensing and interface circuitry in some examples. For instance, a device may include a simple output device that is configured to provide a visual output based on a level of the EDA signal detected by the sensor system. In other examples, however, a wearable device may include processing circuitry configured to process one or more EDA signals to provide enhanced interpretive data associated with a user's sympathetic nervous system activity.

In some examples, a wearable device may include a receiving feature configured to couple to an EDA sensor comprising an integrated electrode pair. The receiving features at different wearable devices may include different form factors that are adapted to the particular wearable device. In some examples, the receiving feature at the wearable device may include electrical contacts for establishing an electrical connection with an EDA sensor. In other examples, a receiving feature may be configured to provide a physical connection only. In some examples, the receiving feature at the wearable device may include one or more retaining elements that are configured to securely house the EDA sensor.

Embodiments of the disclosed technology provide a number of technical effects and benefits, particularly in the areas of electrodermal activity sensors, wearable devices, computing technology, and the integration of electronics with different types of wearable devices. Additionally, one or more aspects of the disclosed technology may address issues that may arise when seeking to provide a practical system and method for incorporating EDA sensors into wearable devices, and providing electronics that are capable of integrating EDA sensors into different types of wearable devices. In accordance with example embodiments of the disclosed technology, an integrated electrode pair can utilize dry electrodes that do not require the use of electrolyte gels required by traditional EDA sensors. Additionally, the electrodes can be formed in concentric formations to increase the sampling distribution area of a user's skin, while also providing an even distribution to increase the signal-to-noise ratio of the sensor. By configuring an inner electrode concentric with an outer electrode, and separating the two electrodes radially by an insulating layer, a more accurate EDA signal can be generated relative to traditional approaches. According to example embodiments, the surface area of the user's skin at the electrode gap between the inner electrode and the outer electrode may not directly depend on electrode pressure. For typical pressures, the area of skin between the electrodes forming the electric gap may stay constant. Additionally, the utilization of concentric electrodes can facilitate a buildup of perspiration under the electrode pair. With such a design, small movements may not therefore substantially move the electrode pair to an area of dry skin. Moreover, a larger and more even distribution of sudoriferous glands can be sampled to increase the signal-to-noise ratio by utilizing a concentric electrode design. Furthermore, the concentric electrodes enable a rotation invariant design, such that small band rotations will not necessarily result in changes in the EDA signal.

Additionally, a single integrated electrode pair can allow for a smaller form factor that can be more aesthetically pleasing and more useful for wearable devices. A single integrated electrode can be easily integrated within a band or other attachment member of a wearable device. The integrated electrode can provide a single contact point for the sensor system to facilitate an efficient and continuous contact between the sensor and user skin. In this manner, reliable and timely measurements of electrodermal activity can be provided without interruptions that are often associated with traditional devices that may require resettling in response to subtle motions. The use of an integrated electrode pair can reduce the overall size of a sensor assembly and thereby provide a more efficient integration with consumer level wearable devices.

In some implementations, in order to obtain the benefits of the techniques described herein, the user may be required to allow the collection and analysis of electrodermal activity or other personal information associated with the user or their device. For example, in some implementations, users may be provided with an opportunity to control whether programs or features collect such information. If the user does not allow collection and use of such signals, then the user may not receive the benefits of the techniques described herein. The user can also be provided with tools to revoke or modify consent. In addition, certain information or data can be treated in one or more ways before it is stored or used, so that personally identifiable information is removed. As an example, a computing system can obtain real-time personal data which can indicate attributes of a user, without identifying any particular user(s) or particular user computing device(s).

With reference now to the figures, example aspects of the present disclosure will be discussed in greater detail.

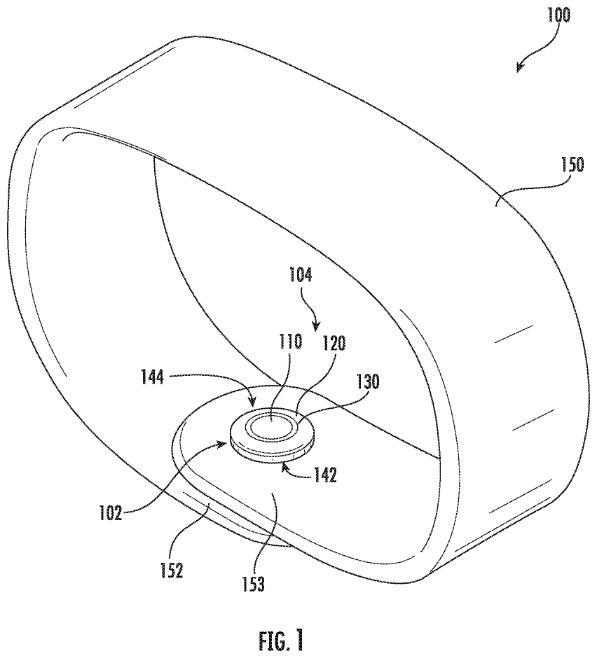

FIG. 1 is a perspective view depicting an example wearable device 100 comprising an electrodermal activity sensor 102 having an integrated electrode pair 104 in accordance with example embodiments of the present disclosure. The integrated electrode pair comprises an inner electrode 110 and an outer electrode 120 that are separated by one or more insulators 130. Integrated electrode pair 104 can be physically coupled to or otherwise integrated with an attachment member 150 to form all or a portion of the wearable device. In FIG. 1, attachment member 150 comprises a band or strap configured to wrap around a wrist, ankle, or other body part of a user when wearable device 100 is one by the user. Attachment member 150 can include a first end 152 and a second end 153 that are joined using a clasp, magnet, or other fastener to form a secure attachment when worn, however many other designs may be used. The strap or other attachment member can be formed from a material such as rubber, nylon, plastic, metal, or any other type of material suitable for attachment to the integrated electrode pair. In some examples, the integrated electrode pair may form part of the clasp as shown in FIG. 1. For example, the integrated electrode pair can form part of a snap or other fastening mechanism that can join the two ends of the strap together when worn by a user. Notably, however, wearable device 100 may take any type or form. For example, rather than being a strap, attachment member 150 could resemble a circular or square piece of material (e.g., rubber or nylon) that can be attached to the integrated electrode pair and substrate material of a wearable device such as a garment. Due to the small form factor and integrated nature of the electrode pair, wearable device 100 can provide a non-obtrusive and effective sensor system for measuring electrodermal activity associated with the user.

By way of example, the integrated electrode pair 104 can include a lower surface 142 that is physically coupled to the attachment member 150 such as the band or strap forming all or part of the wearable device, and an upper surface 144 that is configured to contact the surface of the user's skin in order to measure conductance and/or resistance associated with the skin. The lower surface 142 of the integrated electrode pair can be directly coupled to the attachment member 150 of the wearable device in example embodiments. The integrated electrode pair 104 can be fastened (permanently or removably) to the attachment member, glued to the attachment member, or otherwise physically coupled to the attachment member. In some examples, the lower surface 142 of the integrated electrode pair 104 can be physically coupled to the attachment member 150 or other portion of the wearable device 100 via one or more intervening members.

Sensor 102 is configured to measure electrodermal activity (EDA) associated with users of the wearable device 100. Sensor 102 can include sensing circuitry, processing circuitry, one or more power sources, and/or additional components in various embodiments. The sensor can generate an EDA signal in example embodiments based on electrodermal activity associated with a user using the integrated electrode pair. A visual, audible, and/or haptic output can be provided example embodiments based on the EDA signal. In other examples, data indicative of EDA activity associated with the user can be transmitted from wearable device 100 to a remote computing device.

Inner electrode 110 and outer electrode 120 form concentric EDA electrodes that are attached to or otherwise integrated with wearable device 100. The inner electrode 110 and outer electrode 120 of the integrated electrode pair 104 form at least two concentric electrodes that are separated by insulator 130. Insulator 130 can include one or more one insulating layers. Each of the concentric electrodes can include an upper surface that is configured to contact the skin of a user of the wearable device. When the upper surface 144 of the integrated electrode pair 104 is in contact with the skin of a user of the wearable device 100, the sensor 102 can generate an EDA signal that is indicative of the electrodermal activity associated with the user. In example embodiments, the EDA signal may be indicative of a measurement of conductance or resistance associated with one or more dermal layers of the user's skin. The user's perspiration can provide electrical contact between the integrated electrode pair 104 and the skin surface of the user, thereby enabling ionic flow between inner electrode 110 and outer electrode 120 through one or more dermal layers of the user. A conductance or resistance associated with one or more dermal (e.g., subdermal) layers of the user's skin can then be determined using the integrated electrode pair 104. A current through the user's skin can be measured to determine a measured conductance or resistance in some examples. For instance, a current through the user's skin as coupled through the integrated electrode pair can be measured and correlated to determine conductance, resistance, or another measure indicative of sympathetic nervous system activity.

By utilizing a set of concentric electrodes 110 and 120, a more uniform sampling distribution area adjacent to the upper surface of the insulator 130 can be provided. The surface area of the user's skin at the electrode gap where insulator 130 separates the concentric electrodes may not be directly dependent on electrode pressure in some embodiments. A larger and more even distribution of sudoriferous glands can be sampled through a concentric design as described in order to increase the signal-to-noise ratio associated with the EDA signal. Additionally, due to the buildup of bodily fluids such as perspiration under the integrated electrode pair 104, small movements may not result in movement of the electrode pair to an area of dry skin. For typical pressures, the area of skin may stay constant. In this manner, increased electrode pressure may not affect the diameter of the contact patch between the electrodes and the skin surface, which otherwise may affect the signal to noise ratio of the EDA signal. Furthermore, the use of concentric electrodes 110 and 120 provides a rotation invariant design such that small band rotations may not result in changes to the EDA signal. Additionally, a single integrated electrode pair 104 can provide a smaller form factor that can be more aesthetically pleasing and suitable for a wearable device.

It is noted that while a human being is typically referred to herein, a wearable device as described may be used to measure electrodermal activity associated with other living beings such as dogs, cats, or other animals in accordance with example embodiments of the disclosed technology.

Although principally described with respect to wearable device applications, an EDA sensor including an integrated electrode pair as described herein may be utilized in other applications. For example, an EDA sensor as described may be attached to various objects (e.g., treadmill, bicycle, ball, handle, seat, etc.) with which a user may establish contact.

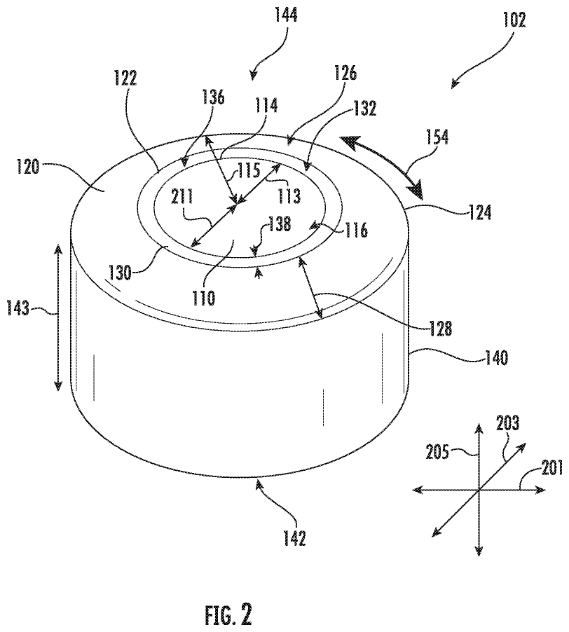

FIG. 2 is a perspective view illustrating additional details of example electrodermal activity sensor 102 in accordance with example embodiments of the disclosed technology. As earlier described, EDA sensor 102 includes an integrated electrode pair 104 comprising an inner electrode 110 and an outer electrode 120 in accordance with example embodiments of the present disclosure. Inner electrode 110 and outer electrode 120 are concentric electrodes in example embodiments. EDA sensor 102 defines a lateral axis 201, a longitudinal axis 203, and a vertical axis 205. EDA sensor 102 has a vertical dimension 143 in a vertical direction parallel to vertical axis 205. Vertical dimension 143 can be defined by a length of a vertical sidewall 140 of the integrated electrode pair 104. EDA sensor 102 has a radial dimension defined by the combined radial dimension of the inner electrode 110, radial dimension 138 of insulator 130, and radial dimension 128 of outer electrode 120.

Inner electrode 110 and outer electrode 120 are separated by an insulator 130 which provides radial separation between the inner electrode and the outer electrode in a radial direction of the concentric elements forming the integrated electrode pair 104. The insulator 130 extends circumferentially around at least a portion of the inner electrode in circumferential direction 154. Outer electrode 120 extends circumferentially around at least a portion of the insulator 130 in circumferential direction 154. The inner electrode is laterally surrounded in its entirety by the outer electrode. The inner and outer electrodes are laterally separated by the insulator. In this manner, an electrode gap 132 is formed that extends in the circumferential direction 154 around inner electrode 110 and in the radial direction between inner electrode 110 and outer electrode 120. The electrode gap matches the outer edge 114 of the inner electrode 110 and the inner edge 122 of the outer electrode 120 in example embodiments. Accordingly, the conductance or resistance of the user's skin can be measured based on current flow that extends radially from the inner electrode 110 to the outer electrode 120 (and/or vice versa) through the user's skin. The current flow can be in multiple radial directions extending 360 degrees around the inner electrode 110 in example embodiments. This can be contrasted with approaches that use two electrodes (bars or circles) that are separated linearly, in which case the conductance or resistance is measured using a current that flows in a linear direction between electrodes.

The inner electrode and outer electrode can be formed with various shapes and dimensions in example implementations. Generally, the inner electrode and the outer electrode are concentric objects or coaxial objects that share the same center or axis. Circles, cylinders, polygons, spheres, polyhedra, and other shapes concentric with one another can be used to form the integrated electrode pair. In some examples, the inner electrode 110, the outer electrode 120, and the insulator 130 may have a generally circular cross-sectional shape as depicted in FIGS. 1 and 2. For instance, the inner electrode 110, the outer electrode 120, and the insulator 130 may have a generally cylindrical shape, with the radius 113 of the insulator 130 being larger than the radius 111 of the inner electrode 110, and the radius 115 of the outer electrode being larger than the radius 113 of the insulating layer. As a specific example, the inner electrode 110 can have a generally cylindrical shape and the insulator 130 can have a generally cylindrical shape with a cylindrical opening in the center or middle of the cylinder to accommodate the inner electrode 110. In this manner, the insulator 130 can extend circumferentially around the inner electrode 110. Similarly, the outer electrode 120 can have a generally cylindrical shape with a cylindrical opening in the center or middle of the cylinder to accommodate the inner electrode 110 and insulator 130. In this matter, the outer electrode 120 can extend circumferentially around the insulator 130 in circumferential direction 154. Insulator 130 can then radially separate the outer electrode 120 from the inner electrode 110.

Various dimensions and sizes of the integrated electrode pair 104 may be used. In FIG. 2, inner electrode 110 has a radial dimension that corresponds directly to its radius 111, which is larger than a radial dimension 138 of the insulator 130. The radial dimension corresponding to radius 111 of the inner electrode is substantially equal to a radial dimension 128 of outer electrode 120. In other examples, the radial dimension of the inner electrode may be different than the radial dimension 128 of the outer electrode. For example, the radial radial dimension of the inner electrode may be larger than radial dimension 128 in some examples. In other examples, radial dimension corresponding to radius 111 of the inner electrode may be less than radial dimension 128. In some examples, radial dimension 138 of insulator 130 may be larger than the radial dimension of the inner electrode and/or the radial dimension 228 of the outer electrode 120.

Various materials may be utilized to form inner electrode 110, outer electrode 120, and the insulator 130 of the integrated electrode pair 104 in various embodiments. In some examples, the electrodes may include one or more metallic materials. The electrodes can be formed from one or more metals and/or metal alloys. As a specific example, the inner electrode 110 and outer electrode 120 may be formed from silver, copper, gold, or alloys of these elements, such as silver chloride. In some examples, the electrodes may include one or more conductive polymers, semiconductors, or other conductive materials. It will be appreciated that any suitable conductive material may be utilized. Insulator 130 can be formed from one or more electrically insulating materials that can electrically insulate the inner electrode from the outer electrode. Any suitable electrically insulating material having high resistivity such as air or space (e.g., a vacuum), nonconductive materials (e.g., plastics, nylons, ceramics, etc.), or semiconducting materials in relatively nonconductive states can be used.

The integrated electrode pair 104 can include a lower surface 142 and an upper surface 144 that define a vertical dimension 143 of the integrated electrode pair. In some embodiments, the upper surface of the outer electrode and the upper surface of the inner electrode can be coplanar. The insulating layer can also be coplanar with the inner electrode and the outer electrode in some embodiments. In such examples, the upper surface of the inner electrode, the upper surface of the outer electrode, and the upper surface of insulating layer can form a common upper surface 144 of the integrated electrode pair that can contact the user's skin.

In some examples, the upper surface 144 of the integrated electrode pair can be substantially flat. In other examples, the upper surface 144 of the integrated electrode pair can include a curve. By way of example, the upper surface of the integrated electrode pair can be convex relative to a user's skin surface in some examples. According to some aspects, the upper surface of the inner electrode and/or the outer electrode may include one or more raised portions that extend vertically relative to other portions of the upper surface. For instance, a ring-shaped raised portion may be formed on the upper surface of the outer electrode at a location that is adjacent to the insulator 130. Additionally or alternatively, a ring-shaped raised portion may be formed on the upper surface of the inner electrode at a location is adjacent to the insulator. The raised portions can help to establish contact between the surface of the user's skin at the electrodes in some examples. Other shapes forming a raised portion for one or more of the electrodes may be used.

A fillet edge can be used for the upper surface of the outer electrode in example embodiments to provide a comfortable surface contact for a user. In some embodiments, the upper surface of the outer electrode and the upper surface of the inner electrode can be coplanar, while the upper surface of the insulating layer includes a vertical offset from the upper surface of the inner electrode and the outer electrode. In some examples, the upper surface of the insulating layer can be recessed relative to the upper surface of the electrodes. In some instances, such an approach may provide an enhanced electrical connection between the electrodes and the user's skin via perspiration that can build up within the recessed area. In other examples, one or more of the inner electrode and/or the outer electrode can be recessed relative to the upper surface of the insulating layer. Such an approach may be useful in situations where the buildup of perspiration may result in an inadvertent short between the inner electrode and outer electrode.

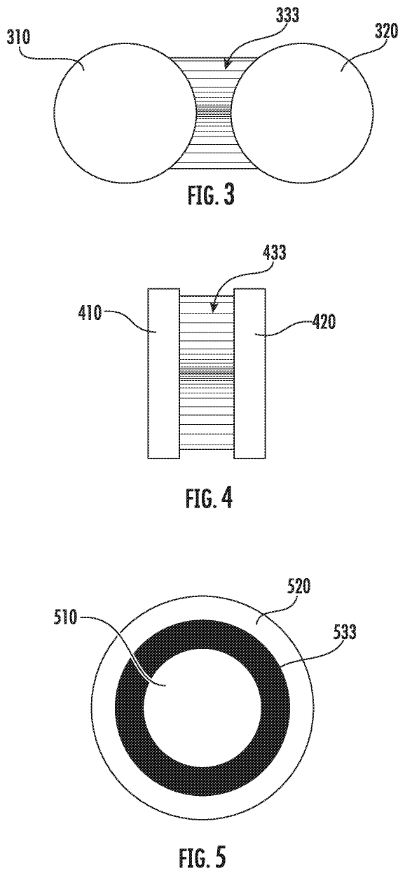

To illustrate example aspects of the disclosed technology, a comparison with example traditional devices is provided. As earlier noted, a goal of electrodermal activity monitoring can be to measure the buildup and excretion of perspiration from eccrine sudoriferous glands (also referred to as sweat glands) as an indicator of sympathetic nervous system (SNS) activity to make inferences about human psychophysiological states. During SNS activation, eccrine sudoriferous glands may excrete perspiration causing skin conductance changes. The signal caused by SNS activation can therefore be measured more effectively by summing the signal across a larger number of sudoriferous glands. Consider an example of a traditional approach that includes two circular electrodes that are separated in a lateral direction. FIG. 3 is a block diagram depicting an example of a bipolar circle pair of electrodes forming a non-integrated structure as may be used by a traditional sensor system. A first electrode 310 can be coupled the surface of a user's skin and separated from a second electrode 320 which is also coupled to the user's skin. The two circular electrodes can sample sweat glands near the electrode pair centerline. As shown by distribution area 333, the two circular electrodes may sample sweat glands less effectively further from the centerline of the electrode pair. An interface between each electrode and a user's skin may sample sudoriferous glands near the electrode pair centerline, which can result in less effective sampling further from the centerline.

Other examples may utilize a set of longer parallel electrodes which may be able to more evenly sample sudoriferous glands between the two electrodes, and thereby increase the coverage of sampled sudoriferous glands. However, such implementations may contact an inconsistent area of the skin. FIG. 4 is a block diagram depicting a parallel bar pair of electrodes of another traditional sensor system. A longer parallel electrode pair can more evenly sample sweat glands between the two electrodes and increase the coverage of sampled sweat glands relative to a pair of circular electrodes. As illustrated, a first electrode 410 is separated linearly from a second electrode 420. The two electrodes may be spaced several millimeters apart in some examples to attempt to evenly sample sweat glands between the two electrodes at a sweat sampling distribution area 433. The use of parallel bars may provide an even sampling of sweat glands between the two electrodes and provide a large coverage area. The two parallel bars, however, may contact an inconsistent area of the skin when implemented with a wearable device. With wearable devices that will be worn as a user moves, the contact between the bars and the user's skin may vary over time, due to small pressure changes and and/or rotational movements, for example.

In some traditional examples that utilize two separate electrode having electrode surfaces that are curved, increasing the electrode pressure can shorten the path that current must pass through the skin, thereby changing an EDA reading. Motion can cause the pressure to change and thereby add noise to an EDA signal. Increasing electrode pressure can affect the diameter of the contact patch, which can increase or decrease parallel current passed through the skin. As such, small movements of the band may induce significant changes in the EDA signals. Moreover, some users have wrist or other body parts that are not circular in profile, for example, that may have a more rectangular shape. For instance, some body parts of some users may have flat areas on the top and inside of the wrist. This can mean that the inside of the wrist may not be a primary point of contact with a band and that electrodes are not pressing into the wrist with constant pressure. In such cases, small movements of electrodes may move into dry areas of skin resulting in a period of time requirement for equilibrium to be reached again. Moreover, the use of a large pair of separate electrodes may result in visible and not aesthetically pleasing designs.

FIG. 5 is a cross-sectional view depicting an integrated electrode pair in accordance with example embodiments of the present disclosure. FIG. 5 depicts an example 500 of an integrated electrode pair comprising an inner electrode 110 and outer electrode 120 separated by an insulator 130. The integrated pair of concentric electrodes can measure the electrodermal activity of a user more evenly, effectively, and/or practically. The integrated electrode pair can provide a more even sudoriferous gland sampling distribution area, while also increasing the coverage area of sampled sudoriferous glands. Additionally, a set of concentric EDA electrodes can provide such benefits while measuring electrodermal activity over a more consistent area of a user's skin.

In this example, an even sweat gland sampling distribution area 533 at the user's skin is provided between the outer electrode 520 and the inner electrode 510. Accordingly, the conductance or resistance of the user's skin can be measured based on current flows extending radially from the inner electrode to the outer electrode (and/or vice versa). This can be contrasted with approaches that use two electrodes (bars or circles) that are separated linearly, in which case the conductance or resistance is measured using a current that flows in a single direction.

Other shapes may be utilized for the inner electrode, the insulating layer, and/or the outer electrode. Generally, any set of two or more concentric objects can be used to form the two electrodes of the integrated electrode pair. By way of example, the electrodes can be formed using spherical-shaped objects, prism-shaped objects (e.g, triangular prism, rectangular prism, or other polygonal prism), cylindrical-shaped objects, etc. It is noted that the electrodes need not have a regular polygonal or polyhedral shape. By way of example, the inner electrode can be formed in a generally cylindrical shape with an outer edge that forms a cloverleaf or other pattern. The outer edge can include multiple arc sections that curve in different directions to increase the length of the outer edge of the inner electrode. By increasing the length of the outer edge of the inner electrode, the sampling area of the user's skin can be increased.

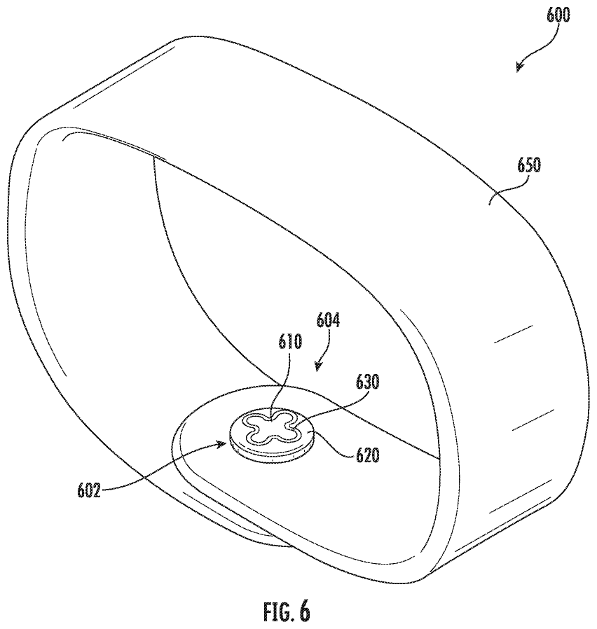

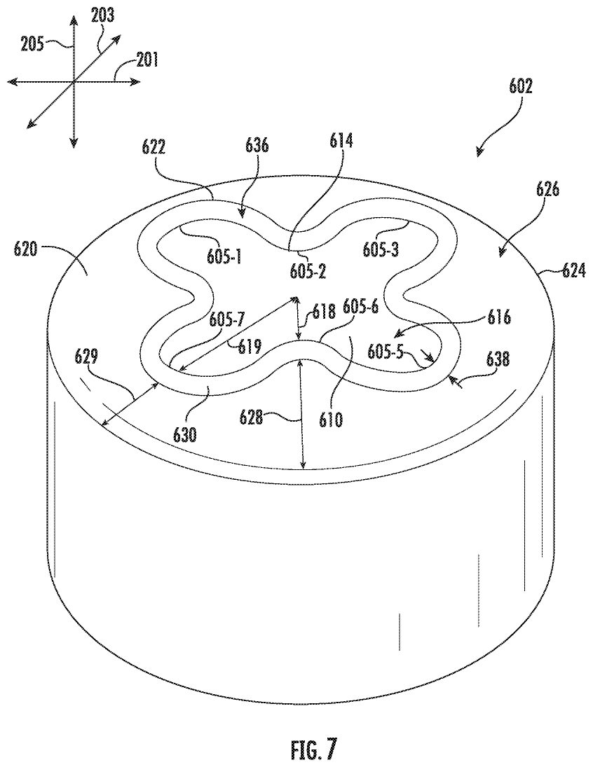

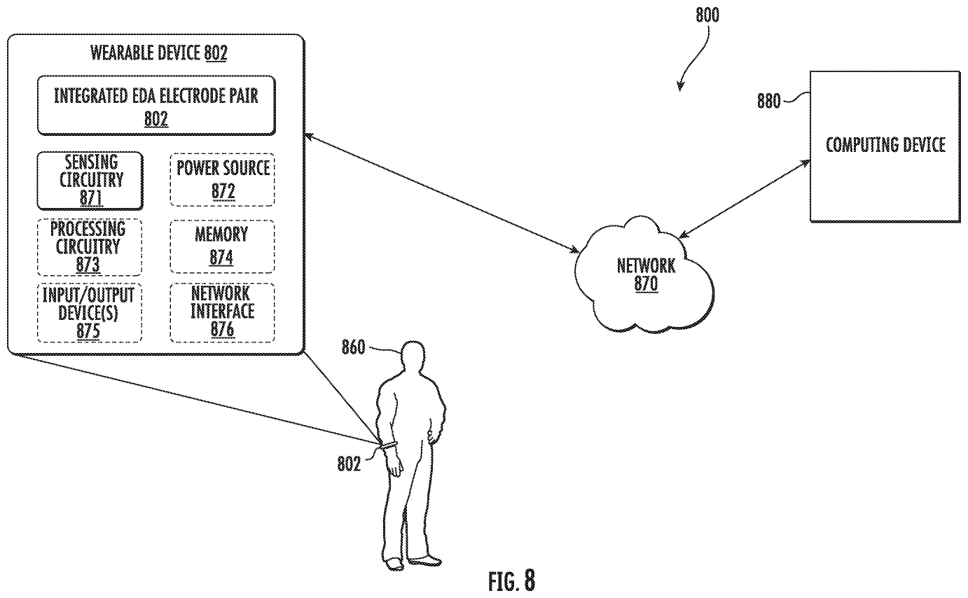

FIG. 6 is a perspective view depicting a wearable device 600 comprising an electrodermal activity sensor 602 having an integrated electrode pair 604 in accordance with example embodiments of the present disclosure where the integrated electrode pair includes a non-circular cross-sectional shape. FIG. 7 is a perspective view depicting additional details of the electrodermal activity sensor depicted in FIG. 6. Inner electrode 610 is formed in a generally cylindrical shape with an outer edge that forms a cloverleaf pattern. It is noted that other patterns may be used. The outer edge 614 of the inner electrode 610 can include multiple arc sections 605-1 to 605-8 that curve in different directions to increase the length of the outer edge of the inner electrode. For example, arc section 605-1 can curve in an opposite direction to arc section 605-5. Additionally, arc section 605-1 can curve in a different direction (e.g., at substantially 90°) relative to arc section 605-2 and arc section 605-8. By increasing the length of the outer edge of the inner electrode, the sampling area of the user's skin can be increased. Each of the multiple arc sections is connected to form a continuous pattern for the outer edge of the inner electrode

The insulator 630 can be formed in a generally cylindrical shape with an opening in the middle. The outer edge 614 of the inner electrode 610 and the outer edge of the insulator 630 can form a matching cloverleaf pattern having a larger radial dimension(s) relative to the inner electrode. The outer electrode 620 can be formed with an inner edge 622 that forms a matching cloverleaf pattern with the outer edge of the insulator 630. The cloverleaf pattern defined by the inner edge of the outer electrode has a larger radial dimension relative to the outer edge of the inner electrode. The inner edge of the outer electrode can be a continuous inner edge. The outer edge of the inner electrode can be a continuous outer edge. The inner electrode is laterally surrounded in its entirety by the outer electrode. The inner and outer electrodes are laterally separated by the insulator. The outer edge 624 of the outer electrode 620 may have a circular shape or other shape (e.g., square, rectangle). The utilization of a cloverleaf or other pattern may increase the sampling area of the user's skin across which a conductance and/or resistance can be measured. Additionally, the use of concentric electrodes with continuous edges enables the generation of an EDA signal based on current flow between the continuous outer edge of the inner electrode and the continuous inner edge of the outer electrode. This current flow may extend radially with an even distribution between electrodes in some embodiments.

Various dimensions and sizes of the integrated electrode pair 604 may be used. In FIG. 7, inner electrode 610 has a radial dimension 618 and a radial dimension 619 that are larger than a radial dimension 638 of the insulator 630. The radial dimension 619 is larger than a radial dimension 629 of outer electrode 120. In other examples, the radial dimensions may be different.

FIG. 8 depicts a block diagram of a wearable device 802 within an example computing environment 800 in accordance with example embodiments of the present disclosure. FIG. 8 depicts a user 860 wearing a wearable device 802. In this example, wearable device 802 is worn around the user's wrist using an attachment number such that an integrated electrode pair 804 of the wearable device 802 is in contact with a surface of the skin of user 860. By way of example, wearable device 802 may be a smartwatch, a wristband, a fitness tracker, or other wearable device. It is noted that while FIG. 8 depicts an example of a wearable device worn around the user's wrist, wearable devices of any form can be utilized in accordance with embodiments of the present disclosure. For instance, integrated electrode pair 804 can be integrated into wearable devices that are coupled to a user in other manners, such as into garments that are worn or accessories that are carried.

FIG. 8 illustrates an example environment 800 that includes a wearable device 802 that is capable of communication with one or more remote computing devices 880 over one or more networks 870. Wearable device 802 can include one or more integrated electrode pairs 804, sensing circuitry 871, processing circuitry 873, input/output device(s) 875 (e.g., speakers, LEDs, microphones, touch sensors), power source 872 (e.g., battery), memory 874 (RAM and/or ROM), and/or a network interface 876 (e.g., Bluetooth, WiFi, USB). Integrated electrode pair 804 can be implemented as an integrated electrode pair 104 or integrated electrode pair 604 as earlier described. Wearable device 802 is one example of a wearable device as described herein. It will be appreciated while specific components are depicted in FIG. 8, additional or fewer components may be included in a wearable device in accordance with example embodiments of the present disclosure.

In environment 800, the electronic components contained within the wearable device 802 include sensing circuitry 871 that is coupled to integrated electrode pair 804 to form an EDA sensor. Power source 872 may be coupled, via one or more interfaces to provide power to the various components of the wearable device, and may be implemented as a small battery in some examples. Power source 872 may be coupled to sensing circuitry 871 to provide power to sensing circuitry 871 to enable the detection of electrodermal activity via integrated electrode pair 804. Power source 872 can be removable or embedded within a wearable device in example embodiments. Sensing circuitry 871 can include various components such as amplifiers, filters, charging circuits, sense nodes, and the like that are configured to sense one or more electrical characteristics of a user via the integrated electrode pair 804. Sensing circuitry 871 can be implemented as voltage sensing circuitry, current sensing circuitry, capacitive sensing circuitry, resistive sensing circuitry, etc. For example, sensing circuitry 871 can cause a current flow between an inner electrode and an outer electrode through one or more layers of a user's skin in order to measure an electrical characteristic associated with the user. In some examples, sensing circuitry 871 can generate an electrodermal activity signal that is representative of one or more electrical characteristics associated with a user of the wearable device. In some examples, an amplitude or other measure associated with the EDA signal can be representative of sympathetic nervous system activity of a user. The EDA signal can include or otherwise be indicative of a measurement of conductance or resistance associated with the user's skin as determined using a circuit formed with the integrated electrode pair. By way of example, the sensing circuitry and integrated electrode pair can induce a current through one or more dermal layers of a user's skin. The current can be passed from one electrode into the user's skin via an electrical connection facilitated by the user's perspiration or other fluid. The current can then pass through one or more dermal layers of the user's skin and out of the skin and into the other electrode via perspiration between the other electrode and the user's skin. The sensing circuitry can measure a buildup and excretion of perspiration from eccrine sudoriferous glands as an indicator of sympathetic nervous system activity in some instances. For example, the sensing circuitry may utilize current sensing to determine an amount of current flow between the concentric electrodes through the user's skin. The amount of current may be indicative of electrodermal activity. The wearable device can provide an output based on the measured current in some examples.

Processing circuitry 873 can include one or more electric circuits that comprise one or more processors such as one or more microprocessors. Memory 874 can include (e.g., store, and/or the like) instructions. When executed by processing circuitry 873, instructions stored in memory 874 can cause processing circuitry 873 to perform one or more operations, functions, and/or the like described herein. Processing circuitry can analyze an EDA signal or other electrical characteristic associated with the user of the wearable device in order to determine data indicative of electrodermal activity of the user. By way of example, processing circuitry 873 can generate data indicative of metrics, heuristics, trends, predictions, or other measurements associated with a user's electrodermal activity.

Wearable device 802 may include one or more input/output devices 875. An input device such as a touch input device can be utilized to enable user to provide input to the wearable device. An output device can be configured to provide a haptic response, a tactical response, an audio response, a visual response, or some combination thereof. Output devices may include visual output devices, such as one or more light-emitting diodes (LEDs), audio output devices such as one or more speakers, one or more tactile output devices, and/or one or more haptic output devices. In some examples, the one or more output devices are formed as part of the wearable device, although this is not required. In one example, an output device can include one or more LEDs configured to provide different types of output signals. For example, the one or more LEDs can be configured to generate patterns of light, such as by controlling the order and/or timing of individual LED activations based on electrodermal activity. Other lights and techniques may be used to generate visual patterns including circular patterns. In some examples, one or more LEDs may produce different colored light to provide different types of visual indications. Output devices may include a haptic or tactile output device that provides different types of output signals in the form of different vibrations and/or vibration patterns. In yet another example, output devices may include a haptic output device such as may tighten or loosen a wearable device with respect to a user. For example, a clamp, clasp, cuff, pleat, pleat actuator, band (e.g., contraction band), or other device may be used to adjust the fit of a wearable device on a user (e.g., tighten and/or loosen).

Network interface 876 can enable wearable device 802 to communicate with one or more computing devices 880. By way of example and not limitation, network interfaces 876 may communicate data over a local-area-network (LAN), a wireless local-area-network (WLAN), a personal-area-network (PAN) (e.g., Bluetooth™), a wide-area-network (WAN), an intranet, the Internet, a peer-to-peer network, point-to-point network, a mesh network, and the like. Network interface 876 can be a wired and/or wireless network interface.

By way of example, wearable device 802 may transmit data indicative of a user's electrodermal activity to one or more remote computing devices in example embodiments. When electrodermal activity is detected by sensing circuitry 871 of the wearable device, data representative of the electrodermal activity may be communicated, via network interface 876, to a remote computing device 880 via network 870. In some examples, one or more outputs of sensing circuitry 871 are received by a microprocessor of processing circuitry 873. The microprocessor may then analyze the output of the sensing circuitry (e.g., an EDA signal) to determine data associated with a user's electrodermal activity. The data and/or one or more control signals may then be communicated to a computing device 880 (e.g., a smart phone, server, cloud computing infrastructure, etc.) via the network interface 876 to cause the computing device to initiate a particular functionality. Generally, network interfaces 876 are configured to communicate data, such as EDA data, over wired, wireless, or optical networks to computing devices.

In some examples, the internal electronics of the wearable device 802 can include a flexible printed circuit board (PCB). The printed circuit board can include a set of contact pads for attaching to the integrated electrode pair 804. In some examples, one or more of sensing circuitry 871, processing circuitry 873, input/output devices 875, memory 874, power source 872, memory 874, and network interface 876 can be integrated on the flexible PCB.

Wearable device 802 can include various other types of electronics, such as additional sensors (e.g., capacitive touch sensors, microphones, accelerometers), output devices (e.g., LEDs, speakers, or micro-displays), electrical circuitry, and so forth. The various electronics depicted within wearable device 802 may be physically and permanently embedded within wearable device 802 in example embodiments. In some examples, one or more components may be removably coupled to the wearable device 802. By way of example, a removable power source 872 may be included in example embodiments.

As a specific example implementation, a wearable device can include an integrated electrode pair 804, sensing circuitry 871, and an output device. Wearable device 802 may generate a visual, audible, and/or haptic output based on a comparison of an EDA signal with one or more thresholds. For instance, an EDA signal that exceeds a threshold may result in a simple output to notify the user of the wearable life

While wearable device 802 is illustrated and described as including specific electronic components, it will be appreciated that wearable devices may be configured in a variety of different ways. For example, in some cases, electronic components described as being contained within a wearable device may at least be partially implemented at another computing device, and vice versa. Furthermore, wearable device 802 may include electronic components other that those illustrated in FIG. 8, such as sensors, light sources (e.g., LED's), displays, speakers, and so forth.

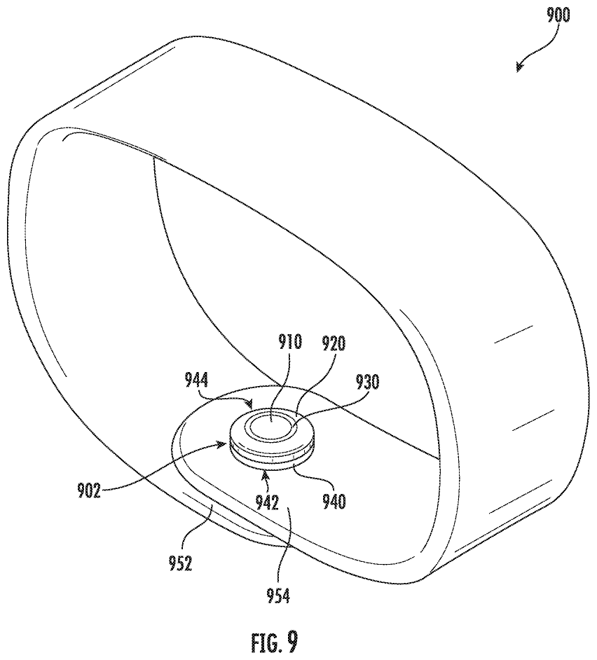

According to some example aspects of the present disclosure, one or more intervening members can be utilized to physically couple an integrated electrode pair to an attachment member of a wearable device. For example, a pressure exertion member can be mounted between the band or other attachment member of a wearable device and the lower surface of the integrated electrode pair in example embodiments. FIG. 9 is a perspective view depicting a wearable device 900 comprising an electrodermal activity sensor 902 having an integrated electrode pair 904 in accordance with example embodiments of the present disclosure. Wearable device 900 further includes pressure exertion member 940 that is configured to maintain pressure between a user's skin and the upper surface 944 of the integrated electrode pair.

In this example, pressure exertion member 940 is mounted between the attachment member 950 of the wearable device and the lower surface 942 of the integrated electrode pair 904. In this manner, pressure exertion member 940 can exert a pressure on the lower surface 942 of the integrated electrode pair 904 and thereby cause a corresponding pressure at the upper surface 944 where the integrated electrode pair 904 contacts the surface of the user's skin. Examples of pressure exertion members include, but are not limited to, springs, compressible materials, etc. Other intervening members may be utilized to couple the integrated electrode pair to a wearable device in example embodiments. In some embodiments, more than one pressure exertion member may be used. For example, a first pressure exertion may be coupled between the lower surface of the inner electrode and the attachment member. The first pressure exertion member can exert a vertical force on the lower surface to enhance contact between the upper surface of the inner electrode and the skin of the user. A second pressure exertion may be coupled between the lower surface of the outer electrode and the attachment member. The second pressure exertion member can exert a vertical force on the lower surface of the outer electrode to enhance contact between the upper surface of the outer electrode and the skin of the user.

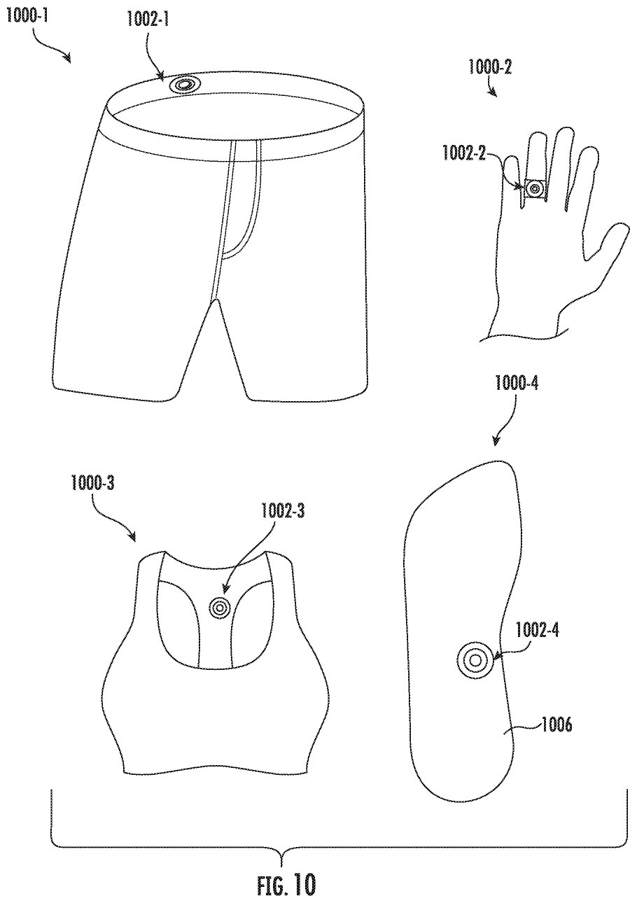

FIG. 10 depicts various examples of wearable devices including an EDA sensor having an integrated electrode pair in accordance with example embodiments. A first example depicts a wearable device 1000-1 comprising a pair of shorts. It will be appreciated that other wearable garments, wearable accessories, and garment containers may implemented in a similar fashion to wearable device 1000-1. Wearable device 1000-1 includes a waistband 1004 to which an EDA sensor 1002-1 including an integrated electrode pair is mounted. EDA sensor 1002-1 can be attached to wearable device 1000-1 utilizing any suitable technique such as by application of a fastener, heat pressing, sewing, gluing or other technique. While application of EDA sensor 1002-1 to waistband 1004 is depicted, it will be appreciated that EDA sensor 1002-1 can be physically coupled to wearable device 1000-1 at other locations.

The second example depicts a wearable device 1000-2 comprising a ring, and more specifically, a ring intended to be worn around the user's finger. It will be appreciated that other wearable accessories such as earrings, bracelets, necklaces, anklets, etc. may be implemented in a similar fashion to wearable device 1000-2. Wearable device 1000-2 includes an attachment member to which EDA sensor 1002-2 is secured, including an integrated electrode pair. EDA sensor 1002-2 can be attached to wearable device 1000-2 utilizing any suitable technique such as by application of a fastener, heat pressing, sewing, gluing or other technique. While application of EDA sensor 1002-2 is positioned in a middle portion of the retaining elements of the ring, it will be appreciated that EDA sensor 1002-2 can be physically coupled to wearable device 1000-2 at other locations.

The third example depicts a wearable device 1000-3 comprising a sports bra. It will be appreciated that other wearable garments may implemented in a similar fashion to wearable device 1000-3. Wearable device 1000-3—includes a strap to which an EDA sensor 1002-3 including an integrated electrode pair is mounted. EDA sensor 1002-3 can be attached to wearable device 1000-3 utilizing any suitable technique such as by application of a fastener, heat pressing, sewing, gluing or other technique. While application of EDA sensor 10020-3 to a strap of the sports bra is depicted, it will be appreciated that EDA sensor 1002-1 can be physically coupled to wearable device 1000-3 at other locations.

The fourth example depicts a wearable device 1000-4 comprising an insole of a shoe. It will be appreciated that other wearable garments may be implemented in a similar fashion to wearable device 1000-4. Wearable device 1000-4 includes a footbed to which an EDA sensor 1002-4 including an integrated electrode pair is mounted. EDA sensor 1002-4 can be attached to wearable device 1000-4 utilizing any suitable technique such as by application of a fastener, heat pressing, sewing, gluing or other technique. While application of EDA sensor 1002-1 to footbed 1006 is depicted, it will be appreciated that EDA sensor 1002-4 can be physically coupled to wearable device 1000-4 at other locations.

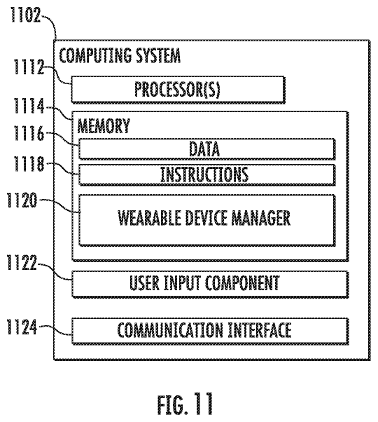

FIG. 11 illustrates various components of an example computing system 1102 that can implement any type of client, server, wearable, and/or other computing device described herein. In embodiments, computing system 1102 can be implemented as one or a combination of a wired and/or wireless wearable device, System-on-Chip (SoC), and/or as another type of device or portion thereof. Computing system 1102 may also be associated with a user (e.g., a person) and/or an entity that operates the device such that a device describes logical devices that include users, software, firmware, and/or a combination of devices. By way of example, computing system 1102 can be used to implement a wearable device or computing device such as computing device 880 as described herein. The computing system 1102 can be a user computing device which can include, for example, a personal computing device (e.g., laptop or desktop), a mobile computing device (e.g., smartphone or tablet), a gaming console or controller, a wearable computing device, an embedded computing device, or any other type of computing device.

The computing system 1102 includes one or more processors 1112 and a memory 1114. The one or more processors 1112 can be any suitable processing device (e.g., a processor core, a microprocessor, an ASIC, an FPGA, a controller, a microcontroller, etc.) and can be one processor or a plurality of processors that are operatively connected. The one or more processors can process various computer-executable instructions to control the operation of computing system 1102 and to enable techniques for, or in which can be embodied a wearable device. Alternatively or in addition, computing system 1102 can be implemented with any one or combination of hardware, firmware, or fixed logic circuitry that is implemented in connection with processing and control circuits. Although not shown, computing system 1102 can include a system bus or data transfer system that couples the various components within the device. A system bus can include any one or combination of different bus structures, such as a memory bus or memory controller, a peripheral bus, a universal serial bus, and/or a processor or local bus that utilizes any of a variety of bus architectures.

The memory 1114 can include one or more non-transitory computer-readable storage mediums, such as RAM, ROM, EEPROM, EPROM, flash memory devices, magnetic disks, etc., and combinations thereof. The memory 1114 can store data 1116 and instructions 1118 which are executed by the processor 1112 to cause the user computing system 1102 to perform operations. Memory 1114 enables persistent and/or non-transitory data storage (i.e., in contrast to mere signal transmission). A disk storage device may be implemented as any type of magnetic or optical storage device, such as a hard disk drive, a recordable and/or rewriteable compact disc (CD), any type of a digital versatile disc (DVD), and the like. Memory 1114 may also include a mass storage media device of computing system 1102.

Computing system 1102 includes a communication interface 1124 that enables wired and/or wireless communication of data 1116 (e.g., received data, data that is being received, data scheduled for broadcast, data packets of the data, etc.). Data 1116 can include configuration settings of the device, media content stored on the device, and/or information associated with a user of the device. Media content stored on computing system 1102 can include any type of audio, video, and/or image data. Computing system 1102 includes one or more data inputs via which any type of data, media content, and/or inputs can be received, such as human utterances, touch data generated by a touch sensor, user-selectable inputs (explicit or implicit), messages, music, television media content, recorded video content, and any other type of audio, video, and/or image data received from any content and/or data source.

Communication interfaces can be implemented as any one or more of a serial and/or parallel interface, a wireless interface, any type of network interface, a modem, and as any other type of communication interface. Communication interfaces provide a connection and/or communication links between computing system 1102 and a communication network by which other electronic, computing, and communication devices communicate data with computing system 1102.

Computer-readable media provides data storage mechanisms to store device data, as well as computer-readable instructions 1118 which can implement various device applications and any other types of information and/or data related to operational aspects of computing system 1102. For example, an operating system can be maintained as a computer application with computer-readable media and executed on processors 1112. Device applications may include a device manager, such as any form of a control application, software application, signal-processing and control module, code that is native to a particular device, a hardware abstraction layer for a particular device, and so on.

Memory 1114 may also include a wearable device manager 1120. Wearable device manager 1120 is capable of interacting with applications and a remote wearable device effective to activate various functionalities associated with computing system 1102 and/or applications through input received from a wearable device. Wearable device manager 1120 may be implemented at a computing device that is local to the wearable device or remote from the wearable device. Wearable device manager 1120 is one example of a controller. In some implementations, wearable device manager 1120 may include one or more electrodermal activity component(s). For example, an electrodermal activity component can store and/or analyze EDA data from a wearable device. In some examples, an EDA activity component can generate data associated with EDA activity such as data for displaying representations of the EDA activity. In some examples, the electrodermal activity component can include detection, classification, prediction or other models that can generate inferences based on detected EDA activity received from a wearable device. In some examples, the EDA component can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks. The EDA component 1020 can be stored in the user computing device memory 1114, and then used or otherwise implemented by the one or more processors 1112.

The computing system 1102 can also include one or more user input components 1122 that receives user input. For example, the user input component 1122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

In some implementations, the computing system 1102 includes or is otherwise implemented by a computing system including one or more computing devices. In instances in which the computing system 1102 is implemented as part of plural server computing devices, such computing devices can operate according to sequential computing architectures, parallel computing architectures, or some combination thereof.

FIG. 11 illustrates one example computing system that can be used to implement the present disclosure. Other computing systems can be used as well.

The technology discussed herein makes reference to servers, databases, software applications, and other computer-based systems, as well as actions taken and information sent to and from such systems. One of ordinary skill in the art will recognize that the inherent flexibility of computer-based systems allows for a great variety of possible configurations, combinations, and divisions of tasks and functionality between and among components. For instance, server processes discussed herein may be implemented using a single server or multiple servers working in combination. Databases and applications may be implemented on a single system or distributed across multiple systems. Distributed components may operate sequentially or in parallel.

While the present subject matter has been described in detail with respect to specific example embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

1. A wearable device comprising:
at least one attachment member; and
an electrodermal activity (EDA) sensor comprising an integrated electrode pair physically coupled to the at least one attachment member, the electrodermal activity sensor configured to provide an EDA signal in response to contact between the integrated electrode pair and a skin surface of a user, the integrated electrode pair comprising at least two concentric electrodes radially separated by at least one insulator, each of the at least two concentric electrodes comprising an upper surface configured to contact the skin surface of the user in order to generate the EDA signal and a lower surface coupled to the at least one attachment member.

2. The wearable device of claim 1, wherein:
the integrated electrode pair comprises a three-dimensional structure having at least one sidewall that extends between the upper surface and the lower surface of the integrated electrode pair.

3. The wearable device of claim 1, wherein:
the at least two concentric electrodes comprise an inner electrode and an outer electrode; and
the insulator extends radially between an outer edge of the inner electrode and an inner edge of the outer electrode.

4. The wearable device of claim 3, wherein:
the inner electrode is laterally surrounded in its entirety by the outer electrode; and
the inner electrode and the outer electrode are laterally separated by the at least one insulator.

5. The wearable device of claim 3, wherein:
the inner electrode comprises a continuous outer edge having a substantially circular shape; and
the outer electrode comprises a continuous inner edge having a substantially circular shape.

6. The wearable device of claim 1, wherein:
the EDA signal provided by the EDA sensor is substantially invariant to rotational movement of the integrated electrode pair relative to the skin surface of the user.

7. The wearable device of claim 3, wherein:
the attachment member comprises a band configured to be worn around a body part of a user of the wearable device;
the lower surface of the inner electrode is physically coupled to the band;
the inner electrode includes at least one substantially vertical sidewall that extends between the upper surface and the lower surface of the inner electrode;
the lower surface of the outer electrode is physically coupled to the band; and
the outer electrode includes at least one substantially vertical sidewall that extends between the upper surface and the lower surface of the outer electrode.

8. The wearable device of claim 3, further comprising:
at least one pressure exertion member coupled between the lower surface of the inner electrode and the band and coupled between the lower surface of the outer electrode and the band;
wherein the at least one pressure exertion member is configured to exert pressure between the integrated electrode pair and the skin surface of the user.

9. The wearable device of claim 3, wherein the upper surface of the inner electrode is coplanar with the upper surface of the outer electrode.

10. The wearable device of claim 3, wherein the upper surface of the inner electrode is vertically separated from the upper surface of the outer electrode.

11. The wearable device of claim 3, wherein the insulator comprises an upper surface that is coplanar with the upper surface of the inner electrode and the upper surface of the outer electrode.

12. The wearable device of claim 3, wherein the insulator comprises an upper surface that is recessed vertically relative to the upper surface of the inner electrode and the upper surface of the outer electrode.

13. The wearable device claim 3, wherein the upper surface of at least one of the inner electrode or the outer electrode is recessed vertically relative to an upper surface of the insulator.

14. The wearable device of claim 3, wherein a sampling distribution area for the integrated electrode pair extends radially between the inner electrode and the outer electrode and extends circumferentially around the inner electrode.

15. The wearable device of claim 3, wherein:
the inner electrode comprises an outer edge having a plurality of arc sections; and
at least two of the plurality of arc sections curve in different angular directions.

16. The wearable device of claim 15, wherein the outer edge of the inner electrode and the inner edge of the outer electrode define a cloverleaf pattern.

17. The wearable device of claim 3, wherein the EDA sensor further comprises:
sensing circuitry communicatively coupled to the inner electrode and the outer electrode, the sensing circuitry configured to generate the EDA signal;
the EDA signal is representative of a measure of at least one of a conductance or a resistance associated with at least one skin layer of the user; and
the sensing circuitry is configured to provide an indication of sympathetic nervous system (SNS) activity of the user based at least in part on the measure of at least one of the conductance for the resistance associated with the at least one skin layer.

18. The wearable device of claim 1, wherein:
the wearable device comprises a wearable garment.

19. An electrodermal activity (EDA) sensor, comprising:
an inner electrode including a lower surface and an upper surface separated by at least one sidewall;
an outer electrode concentric with the inner electrode, the outer electrode having a larger radius than the inner electrode and including a lower surface and an upper surface separated by at least one sidewall;
one or more insulating materials concentric with the inner electrode and the outer electrode, the one or more insulating materials radially separating the inner electrode from the outer electrode and electrically insulating the inner electrode from the outer electrode; and
sensing circuitry communicatively coupled to the inner electrode and the outer electrode, the sensing circuitry configured to generate an EDA signal in response to contact between an outer surface of the inner electrode and a skin surface of a user and contact between an outer surface of the outer electrode and the skin surface of the user.

20. A method of determining electrodermal activity (EDA) associated with a user of a wearable device, comprising:
generating an EDA signal in response to contact between an integrated electrode pair and a skin surface of a user, the integrated electrode pair comprising at least two concentric electrodes radially separated by a least one insulator, each of the at least two concentric electrodes comprising an upper surface that is configured to contact the skin surface of the user in order to generate the EDA signal and a lower surface that is coupled to at least one attachment member; and
determining a measure of electrodermal activity associated with the user in response to the EDA signal.
